(19) United States
(12) Patent Application Publication
(10) Pub. No.: US 2026/0108716 A1
(43) Pub. Date: Apr. 23, 2026

(54) Replaceable Makeup Ink Cartridge System

(71) Applicant: Trina Ireland, Fruitland, ID (US)

(72) Inventor: Trina Ireland, Fruitland, ID (US)

(21) Appl. No.: 18/923,532

(22) Filed: Oct. 22, 2024

(51) Int. Cl. A61M 37/00

(52) U.S. Cl. CPC A61M 37/0076

(57) ABSTRACT

A replaceable makeup ink cartridge system is provided which may utilize a distal applicator portion at least partially enclosed by a central gripping portion. The distal applicator portion may utilize a needle cartridge housing which itself may have a plurality of apertures disposed therethrough. The needle cartridge housing may enclose a reciprocating slide element utilizing a makeup ink cartridge therein. The central gripping portion may have a tapered outer housing defining a cavity which has a driving element disposed therein and is in operable communication with the reciprocating slide element. The system may further utilize an applicator actuator in electrical communication with the driving element and electrical or mechanical communication with locking tabs securing the distal applicator portion in place.

### BACKGROUND OF THE INVENTION

The makeup industry, particularly in the realm of permanent and semi-permanent makeup applications such as microblading and tattooing, has seen continued technological advancements over the years. Traditionally, these applications required manual adjustments of ink flow and relied heavily on the skill and precision of the makeup artist. This sector has burgeoned into a multi-million dollar industry that demands continual innovation to improve precision, reduce waste, and enhance user comfort and safety.

Despite advancements, there remains a gap in the market for systems that offer both versatility and precision in ink delivery. Specifically, existing solutions often suffer from issues such as inconsistent ink flow, lengthy setup and cleanup times, and limited compatibility between different types of inks and machines. These gaps in the industry present opportunities for development in creating more adaptable and efficient makeup delivery systems.

Reviews of existing makeup ink cartridge systems reveal that while many such devices aim to simplify the ink application process, they often fail to address the full range of user needs. A persistent issue in the makeup ink cartridge industry is the difficulty artists face when attempting to switch between different ink types during a session. This process is often cumbersome and time-consuming, requiring a complete system cleanup before a new ink type can be introduced. This not only interrupts the workflow but also limits the artist's ability to blend colors or switch between ink types based on immediate client needs or creative changes.

Another significant concern is maintaining sterility throughout the makeup application process. Traditional systems can expose inks and equipment to contaminants each time a cartridge is opened, changed or refilled. This poses a high risk of infection to clients, which is a serious drawback in environments where skin integrity is compromised, such as during tattooing or microblading procedures. Contaminants could also cause the makeup applicator to clog, malfunction, or otherwise become less efficacious.

Furthermore, controlling the exact amount of ink dispensed during application remains a challenge with existing systems. Inaccurate ink flow can lead to uneven application, waste of expensive inks, and potential dissatisfaction from clients. Precise control over ink flow is crucial for achieving high-quality results in detailed cosmetic applications, and any inconsistency can significantly affect the outcome of the makeup or tattoo being applied.

The prevailing challenges underscore the need for an innovative solution that can provide ease of use, improved hygiene, and adaptability in ink selection. A device like the replaceable makeup ink cartridge system proposes an integrated approach by featuring replaceable cartridges that are both easy to install and remove, and capable of precise ink flow control, potentially revolutionizing the market by addressing these longstanding issues.

Such a system could substantially enhance operational efficiency, reduce cross-contamination risks, and provide artists with the ability to easily customize color mixes and makeup application density. This aligns with the industry's move towards more cost-effective, client-specific, versatile, and hygienic solutions in makeup application technologies.

### BRIEF DESCRIPTION OF THE DRAWINGS

FIG. 1A illustrates an elevational cross-sectional view of a replaceable makeup ink cartridge system in accordance with some embodiments of the present invention.

FIG. 1B illustrates a detailed elevational cross-sectional view of a gripping portion of a replaceable makeup ink cartridge system in accordance with some embodiments of the present invention.

FIG. 1C illustrates a detailed elevational cross-sectional view of an applicator portion of a replaceable makeup ink cartridge system in accordance with some embodiments of the present invention.

FIG. 2 illustrates a perspective view of a replaceable makeup ink cartridge system with a makeup ink cartridge in a state of partial insertion within a body of the replaceable makeup ink cartridge system in accordance with some embodiments of the present invention.

FIG. 3A illustrates a perspective view of a replaceable makeup ink cartridge system with an applicator portion in a coupled configuration in accordance with some embodiments of the present invention.

FIG. 3B illustrates a perspective view of a replaceable makeup ink cartridge system with an applicator portion in a decoupled configuration in accordance with some embodiments of the present invention.

FIG. 4 illustrates a detailed partial cross-sectional view of an applicator actuator of a replaceable makeup ink cartridge system in accordance with some embodiments of the present invention.

### DETAILED DESCRIPTION OF THE INVENTION

Before describing the present invention in detail, it is to be understood that the invention is not limited to any one of the particular embodiments, which of course may vary. It is also to be understood that the terminology used herein is for the purpose of describing particular embodiments only, and therefore is not necessarily intended to be limiting. As used in this specification and the appended claims, terms in the singular and the singular forms “a,” “an,” and “the” include plural referents unless the context clearly dictates otherwise. Thus, for example, reference to “a replaceable makeup ink cartridge system” or any of its constituent parts also includes a plurality of replaceable makeup ink cartridge systems or any of its constituent parts and the like.

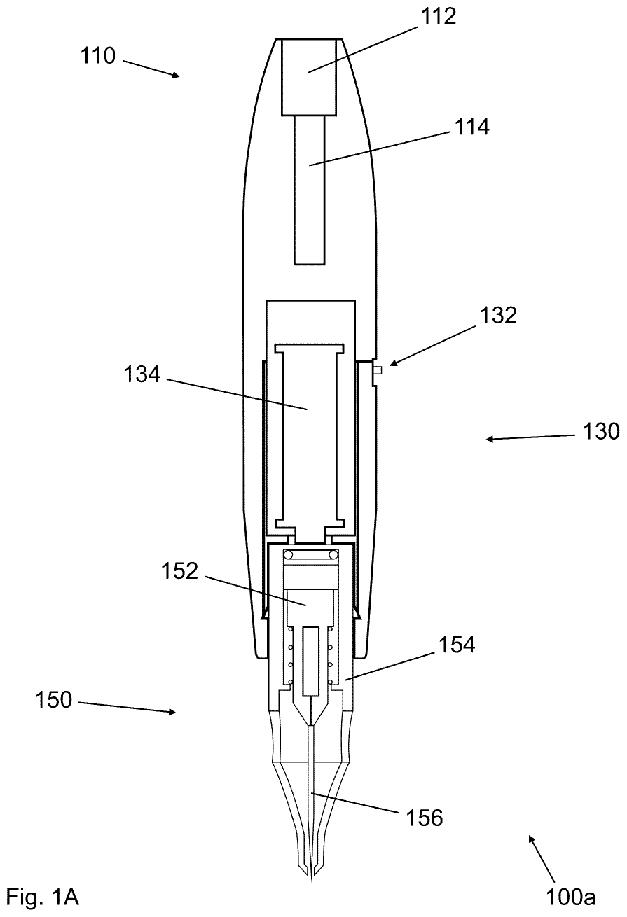

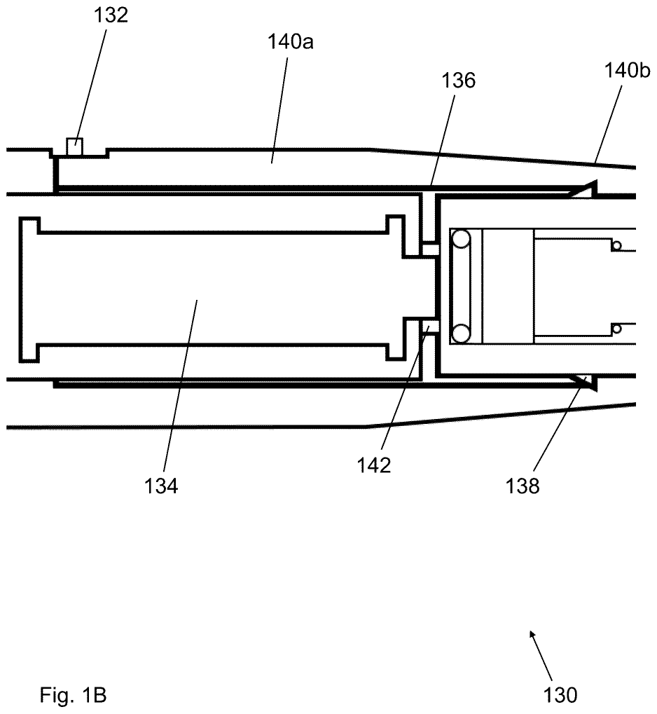

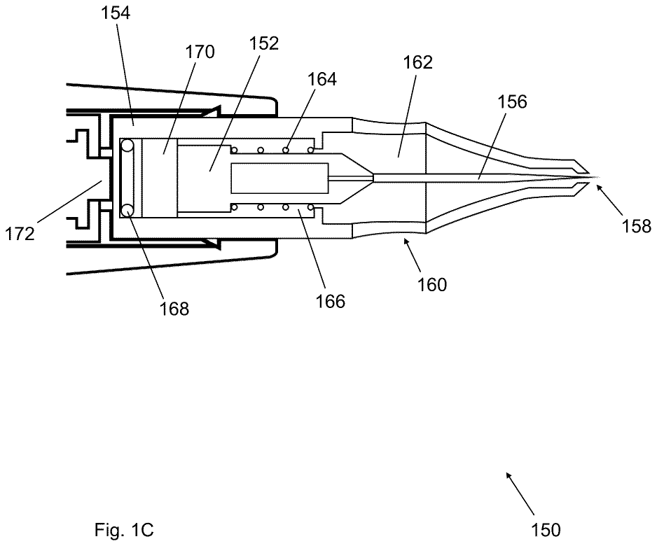

Exemplary embodiments of the present invention are illustrated in the accompanying figures. As shown in FIG. 1A, an elevational cross-sectional view of a replaceable makeup ink cartridge system 100a is provided. The replaceable makeup ink cartridge system 100a may comprise a proximal attachment portion 110, a central gripping portion 130, and a distal applicator portion 150. The central gripping portion 130 may be disposed between the proximal attachment portion 110 and the distal applicator portion 150.

The proximal attachment portion 110 may comprise a charging port 112 and a thermal regulation element 114. The central gripping portion 130 may comprise an applicator actuator 132 electrically coupled to a driving element 134. The distal applicator portion 150 may comprise a reciprocating slide element 152, a needle cartridge housing 154, and an applicator needle 156. The reciprocating slide element 152 may be driven by the driving element 134 which causes the applicator needle 156 to reciprocate and apply a solution therefrom.

As shown in FIG. 1B, a detailed elevational cross-sectional view of a gripping portion 130 of a replaceable makeup ink cartridge system is provided. In one embodiment, the gripping portion 130 of the replaceable makeup ink cartridge system may comprise an applicator actuator 132 that is in operable, e.g. electrical, magnetic and/or mechanical, communication with a driving element 134, a release actuator 136, and a plurality of locking tabs 138. All of these structural components may be fully or partially enclosed within a first housing portion 140a and a second housing portion 140b.

The first housing portion 140a may comprise a generally cylindrical shape with no degree of taper, whereas the second housing portion 140b may comprise a generally conical tapered shape. The first and second housing portions 140a, 140b may collectively comprise an internal aperture 142 formed therein which allows the driving element 134 to provide an impulse to a driving magnet of the distal applicator portion 150 of FIG. 1A.

As shown in FIG. 1C, a detailed elevational cross-sectional view of an applicator portion 150 of a replaceable makeup ink cartridge system is provided. In one embodiment, the applicator portion 150 of the replaceable makeup ink cartridge system may comprise a reciprocating slide element 152, a needle cartridge housing 154, and an applicator needle 156 terminating at a needle point 158. Further, the needle cartridge housing 154 may comprise an aperture 160 disposed therethrough which exposes a cavity 162 therein.

The reciprocating slide element 152 may be at least partially surrounded by a coil or spring 164 and an encasement medium 166. A driving magnet 170 may be disposed between the reciprocating slide element 152 and the driving element 134 of FIG. 1B. Additionally, a gasket 168 is also situated between the driving magnet 170 and the driving element 134 of FIG. 1B. An impulse delivery portion 172 of the driving element of FIG. 1B may be disposed adjacent the gasket 168 in order to provide the reciprocating slide element 152 with periodic impulses via the driving magnet 170 in order to drive the reciprocating motion of the needle 156.

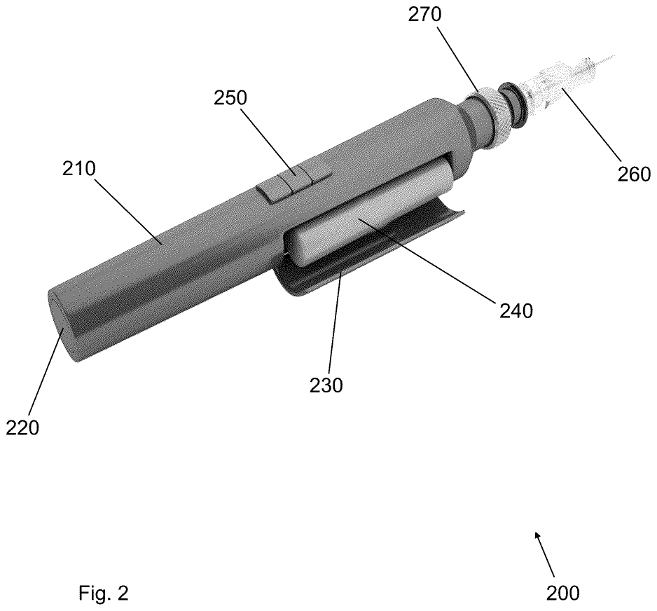

As shown in FIG. 2, a perspective view of a replaceable makeup ink cartridge system 200 with a makeup ink cartridge 240 in a state of partial insertion within an elongate body 210 of the replaceable makeup ink cartridge system 200 is provided. The replaceable makeup ink cartridge system 200 may comprise the elongate body 210 having a replaceable rechargeable battery 220 at a proximal end portion thereof. A central gripping portion of the elongate body 210 may comprise a partially removable hatch door 230 that rotates about a hinge coupled to the elongate body 210 in order to allow access to the makeup ink cartridge 240.

The makeup ink cartridge 240 is illustrated in FIG. 2 as being a state of partial insertion. When the hatch door 230 is disposed in a fully open state, the makeup ink cartridge 240 may be accessible to a user to remove or insert the cartridge 240 relative a cavity within the elongate body 210 as shown in FIG. 2. When the hatch door 230 is disposed in a fully closed state where the door 230 is flush with the remainder of the alginate body 210, the makeup ink cartridge 240 may not be accessible to a user and may be fully engaged with a driving element and reciprocating slide element to deliver makeup ink to the user via a needle cartridge 260. When the makeup ink cartridge 240 is in the fully engaged configuration, it is aligned in parallel with the elongate body 210 along a common axis arranged axially along the lengths of the cartridge 240 and body 210.

An applicator actuator 250 may be disposed within an outer surface of the central gripping portion of the elongate body 210. The applicator actuator 250 may comprise a plurality of actuator buttons arranged in series that allow the user to change the mode of function of the needle cartridge 260 via the driving element. The various actuator buttons may activate reciprocation of the needle within the needle cartridge 260, increase the frequency of reciprocation of the needle within the needle cartridge 260, decrease the frequency of reciprocation of the needle within the needle cartridge 260, cease reciprocation of the needle within the needle cartridge 260, release the needle cartridge 260 from the remainder of the replaceable makeup ink cartridge system 200, and the like or any combination thereof.

The locking collar 270 depicted in FIG. 2 of the replaceable makeup ink cartridge system 200 may serve a dual purpose in the operational functionality of the system 200. Firstly, the locking collar 270 may act as a critical component for securing the needle cartridge 260 in place, ensuring it remains aligned and stable during use. By twisting the locking collar 270, users can easily release or secure the needle cartridge 260, facilitating quick changes and maintenance. Alternatively, the locking collar 270 may be translated axially along the length of the replaceable makeup ink cartridge system 200 without any rotation in order to release the needle cartridge 260 therefrom. In some embodiments, the actuator button ceasing reciprocation of the needle cartridge 260 or placing it in a condition for release may need to be actuated in order for the locking collar 270 to be able to release the needle cartridge 260.

Secondly, the locking collar 270 may be ergonomically designed to serve as an additional gripping point for the user during use. Specifically, the locking collar 270 may comprise a textured embossed pattern structure disposed on an outer surface of the collar 270. Further, the locking collar 270 may comprise a general cylindrical ridge structure. These design features allow users to grip the elongate body 210 more securely and comfortably, while providing enhanced control over the needle cartridge 260 and the makeup application process more generally. This dual functionality of locking and grip enhancement of the locking collar 270 not only improves the usability of the replaceable makeup ink cartridge system 200 but also enhances the precision and safety of the makeup application.

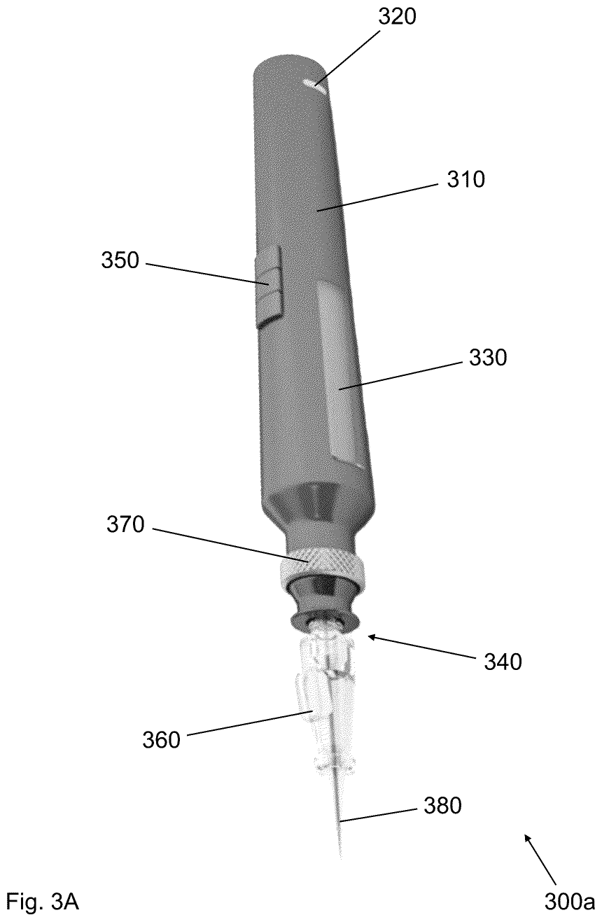

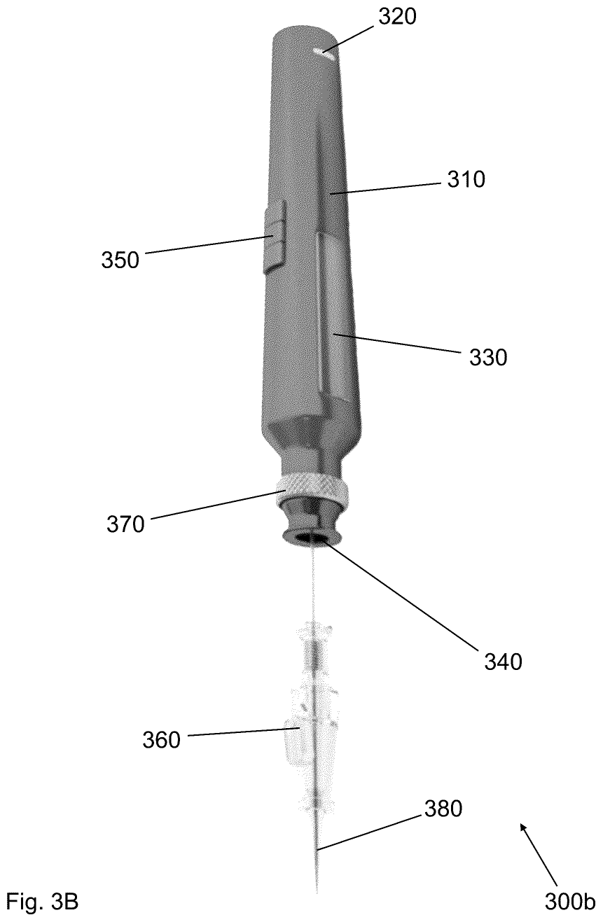

As shown in FIG. 3A, a perspective view of a replaceable makeup ink cartridge system 300a with an applicator portion 360 in a coupled configuration is provided. The replaceable makeup ink cartridge system 300a may comprise an elongate body 310 having a charging port 320 disposed at a distal end thereof. An elongate compartment door 330 may be disposed along the length of the alginate body 310 as shown in FIG. 3A. The elongate body 310 may terminate at a coupling point 340 which may comprise a peripheral flange disposed around a central aperture aligned axially along the length of the elongate body 310.

The aperture of the coupling point 340 may be shaped to accept the applicator portion 360 therein in an iterative removable manner. In order to effectuate locked insertion or free removal of the applicator portion 360 to or from the coupling point 340, either or both of an applicator actuator 350 and a locking collar 370 may be utilized. The locking collar 370 may be designed to either rotate around or translate along the axial axis, can effectively lock the applicator portion 360 into place or release it for removal. By twisting or sliding the locking collar 370, users can engage or disengage a series of mechanical locks or clasps that interact with corresponding features on the applicator portion 360, thereby ensuring a secure connection when in the locked position and an easy removal process when unlocked.

The mechanics of the locking mechanism involving the locking collar 370 may operate through a combination of mechanical interactions that ensure the secure fastening of the applicator portion 360 at the coupling point 340. This removable interconnection system may include a bayonet mount style, where the collar 370 features slots that align with pins on the applicator portion 360. By rotating the collar 370, the pins slide into locked positions along a guided track, which provides a firm and secure lock. In alternative embodiments, the removable interconnection mechanism could involve a threaded interface where the collar 370 screws onto the applicator portion 360, using threads to tighten and secure the connection. This type of threaded approach ensures that the applicator portion 360 can withstand operational forces without loosening inadvertently.

In other embodiments, the locking collar 370 may also employ a cam-lock mechanism where a cam within the collar 370 rotates against a wedge or lip on the applicator portion 360, creating a clamping force that holds the components together securely. The translation motion of the collar 370, either pushing forward or pulling back, might activate a spring-loaded pin that locks into place, providing a quick-release function that is both secure during operation and easy to disengage when necessary. These mechanical configurations are engineered to ensure that the locking and unlocking actions are both intuitive and require minimal effort, enhancing the user's efficiency and comfort during use.

As shown in FIG. 3B, a perspective view of a replaceable makeup ink cartridge system 300b with an applicator portion 360 in a decoupled configuration is provided. The replaceable makeup ink cartridge system 300b may include an elongate body 310, featuring a charging port 320 at its distal end. Along the length of the elongate body 310, an elongate compartment door 330 is visible. This elongate body 310 terminates at a coupling point 340, characterized by a peripheral flange surrounding a central aperture, which is axially aligned with the elongate body 310.

The central aperture of the coupling point 340 is specifically shaped to accept the applicator portion 360 in a manner that allows for iterative removal. The applicator portion 360 is shown in FIG. 3B as being in a decoupled state, facilitated by the interaction with either or both of the applicator actuator 350 and the locking collar 370. The locking collar 370, which can rotate or translate along the axial axis, plays a pivotal role in securely locking the applicator portion 360 in place or allowing its removal. By adjusting the locking collar 370, via either twisting and/or sliding, users can activate or deactivate mechanical locks or clasps that securely engage with the applicator portion 360, ensuring a stable yet detachable connection.

The locking mechanism of the collar 370 is intricately designed with several mechanical strategies to ensure secure fastening of the applicator portion 360 at the coupling point 340. This includes a bayonet mount mechanism where slots on the collar 370 align with and slide over pins on the applicator portion 360, securing it into place. Alternatively, a threaded interface may be employed, allowing the collar 370 to screw onto the applicator portion 360 and tighten securely, capable of withstanding the operational forces applied during use.

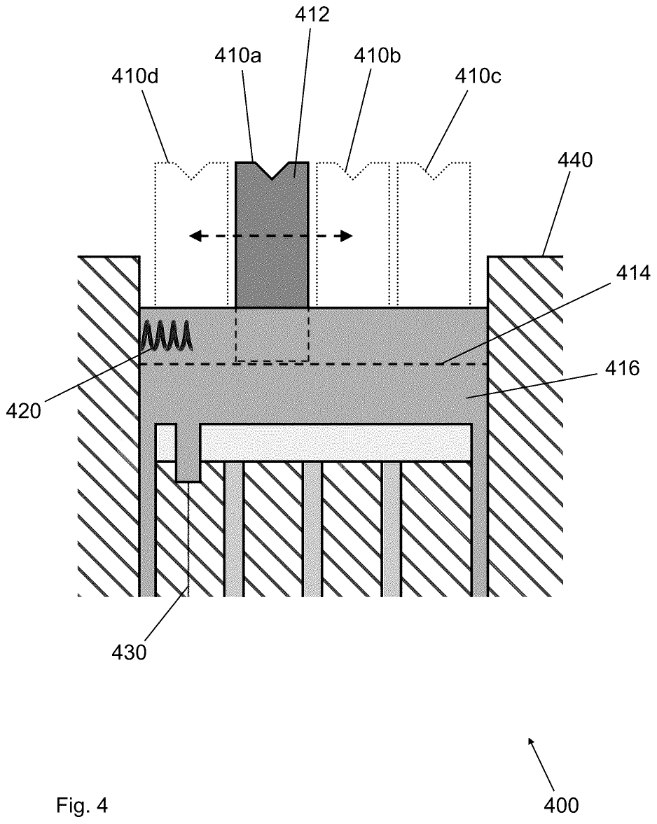

As shown in FIG. 4, a detailed partial cross-sectional view of an applicator actuator 400 of a replaceable makeup ink cartridge system is provided. In some embodiments, the applicator actuator 400 may comprise a slide handle 412 that may be iteratively disposed across a plurality of positions 410a-410d within an actuator housing 416 which may be fixed within the replaceable makeup ink cartridge system housing 440 as illustrated in FIG. 4. The slide handle 412 may translate iteratively along a track 414 between the plurality of positions 410a-410d. When the slide handle 412 is disposed in a release position 410d, the slide handle 412 may be disposed adjacent a buffer spring 420. When the handle 412 compresses the spring 420, the slide handle 412 may be transiently disposed within the release position 410d such that a communication signal is generated along transmission line 430 that may release a locking mechanism holding the applicator portion 360 of FIGS. 3A and 3B in place, thereby allowing the applicator portion 360 to be removed from the coupling point 340 as described.

In some embodiments of FIGS. 1A-4, the elongate body of the replaceable makeup ink cartridge system may take the form of a pen body which exemplifies advanced ergonomic engineering to maximize user comfort and operational efficiency. The body may be shaped with a contoured grip that molds to the natural curvature of the hand, significantly reducing fatigue and enhancing stability during extended use. This ergonomic focus may be complemented by an intelligent internal layout where the power source and motor are strategically placed to achieve optimal weight distribution, thereby facilitating ease of handling and precise control during the makeup application process.

Additionally, the pen body may be equipped with specialized interface ports that ensure seamless integration with replaceable components such as the ink cartridge and the power source. These ports are designed to prevent misalignment and errors in connectivity, promoting a reliable and smooth operation. The choice of materials for the pen body reflects a commitment to durability and hygiene, with surfaces that are easy to clean and maintain, such as rubberized textures that offer a firm grip without compromising on the device's overall hygiene. Moreover, user-friendly controls are thoughtfully positioned to be easily accessible, enabling quick adjustments without the need to alter the grip, thus maintaining focus and efficiency during intricate makeup applications. These features collectively underscore the system's design philosophy, which prioritizes user comfort and functional harmony.

In some embodiments of FIGS. 1A-4, the replaceable makeup ink cartridge system incorporates several innovative features to enhance user convenience and system functionality. The design includes magnetic couplings that secure the cartridge in place effortlessly, facilitating easy, tool-free insertion and removal, complemented by a tactile feedback mechanism that confirms correct placement. This system may be adaptable, allowing for both disposable and reusable cartridges, which may include a visual indicator to display the ink color.

Additionally, the cartridge features a self-sealing mechanism to prevent ink spills and preserve unused ink, supporting sustainable usage practices. The precise alignment may be ensured by physical guides on both the cartridge and the pen body, enhancing reliability and performance. To further protect the ink quality, a specialized vacuum-sealed compartment within the pen body prevents air exposure and ink drying, maintaining the integrity of the ink over time.

Operational efficiency may be supported by a lock-and-release lever, which allows for secure placement and easy removal of the cartridge, minimizing the risk of mess during changes. This lever may be designed for multifunctional use, providing various operational modes including different needle speeds and ink flow settings, thereby accommodating a wide range of application needs. The cartridge system is also designed for refillability, featuring a self-sealing valve, a screw cap with a gasket for secure closure, and a pressure balancing system to prevent leaks, ensuring durability and repeated usability.

In some embodiments of FIGS. 1A-4, the reciprocating needle within the replaceable makeup ink cartridge system plays a pivotal role in the precise application of makeup inks. Integrated seamlessly with the ink cartridge, it allows for the continuous flow of ink, aligning perfectly with the cartridge's outlet to prevent leakage and ensure steady application. The needle's movement may be powered by a meticulously designed electric motor located within the pen body, which ensures that the needle operates with exceptional precision. The motor's speed and the needle's depth are adjustable via user-friendly actuators, providing customized control over the application process, catering to varying skin types and design intricacies.

Moreover, the electric motor's integration with the system's wireless power source affords it a reliable and steady operation, important for maintaining consistent performance during extended use. This configuration not only optimizes the needle's functionality for precise ink delivery but also includes features that allow for the regulation of ink flow, adapting to specific application needs. The entire assembly may be constructed from materials that are capable of being sterilized, adhering to the highest standards of hygiene required in skin application procedures, ensuring safety and cleanliness in every use.

In some embodiments of FIGS. 1A-4, the wireless power source in the replaceable makeup ink cartridge system may be engineered to seamlessly integrate with the electric motor, supplying the necessary power for the precise and continuous operation of the reciprocating needle. This integration ensures that the system operates reliably over extended periods, supported by advanced battery management that optimizes power delivery and enhances charging efficiency. This setup allows for consistent performance essential for professional makeup applications.

Additionally, the system may be equipped with user-friendly actuators, including an ON/OFF switch and ink flow speed controls, which are strategically placed to enhance user interaction with the device. These actuators allow users to easily adjust the operational parameters of the system, such as turning it on or off and regulating the speed at which ink may be applied. This control over the ink flow speed may be crucial, as it directly affects the needle's movement speed and the rate at which ink may be dispensed from the cartridge, enabling precise and customizable makeup ink application.

In some embodiments of the FIGS. 1A-4, the material choices in the replaceable makeup ink cartridge system are critical for ensuring durability, safety, and ergonomic comfort. The pen body may be crafted from high-grade plastics or metal alloys, each selected to balance durability and weight for optimal user comfort. Metals provide a more robust and premium feel, while plastics offer cost-effectiveness and flexible design options. For the ink cartridge, biocompatible materials are utilized to ensure safety, as the ink directly contacts the skin. These materials are specifically chosen to be non-reactive and hypoallergenic, minimizing the risk of skin irritation. Additionally, the needle may be made from surgical-grade stainless steel, providing the necessary strength to maintain sharpness and precision, and may be designed to be disposable to uphold hygiene standards without the need for sterilization between uses.

In some embodiments of FIGS. 1A-4, the functional design of the replaceable makeup ink cartridge system may be engineered to support operational excellence and user comfort. The pen body serves as the structural backbone, providing a robust yet ergonomic housing that ensures stability and ease of use during makeup applications. The wireless power source may be integral, offering a consistent and reliable energy supply that enables the electric motor to operate the reciprocating needle without interruptions. This needle, pivotal for ink delivery, moves with precision and efficiency, ensuring high-quality ink application directly to the skin.

Additionally, the electric motor's role may be essential, as it directly influences the needle's performance by adjusting its speed and precision, thereby affecting the overall quality of the ink application. Actuators within the system, including the ON/OFF switch and ink flow speed adjuster, are strategically placed to provide users with intuitive control over the device's operations, enhancing the ease of use and ensuring optimal functionality. The ink cartridge, designed for easy accessibility, allows for quick ink color changes or refilling, which supports seamless transitions during varied makeup sessions, maintaining efficiency and enhancing user experience.

In some embodiments of FIGS. 1A-4, the replaceable makeup ink cartridge system introduces a temperature-controlled ink feature, significantly enhancing ink flow and skin application quality under various environmental conditions. By maintaining the ink temperature slightly above room temperature, specifically within the 30-35° C. (86-95° F.) range, the system ensures optimal ink viscosity for smooth flow and consistent application, reducing the risk of skin irritation, particularly in sensitive areas. This temperature regulation adapts to different ambient temperatures, ensuring reliable performance regardless of external conditions.

Additionally, the replaceable makeup ink cartridge system may include a color mixing cartridge designed to allow users to create custom ink shades by mixing primary colors. This may be facilitated by microfluidic channels and precision pumps within the cartridge, which precisely control the flow and mixing of different inks. A dedicated mixing chamber within the cartridge ensures that the inks are thoroughly blended before being delivered to the needle, optimizing color accuracy and consistency. This innovative feature expands the palette of available colors, enhancing the flexibility and creativity of makeup applications.

In some embodiments of FIGS. 1A-4, the replaceable makeup ink cartridge system introduces an eco-friendly, refillable cartridge design aimed at minimizing environmental impact while ensuring high-quality makeup application. This design features cartridges made from durable, non-toxic materials that are engineered to withstand repeated use without degrading. A secure, leak-proof refill mechanism facilitates easy ink replenishment, preventing contamination and maintaining hygiene. Standardized containers for eco-friendly ink ensure compatibility and ease of use, with inks formulated from natural or organic pigments that are safe for both skin and environment.

Additionally, the replaceable makeup ink cartridge system may incorporate a cartridge cleaning feature to ensure hygienic reuse of the cartridges. To further enhance the user experience and client comfort, the cartridges can be infused with skin soothing agents such as Aloe Vera, Vitamin E, Allantoin, Tea Tree Oil, and Chamomile Extract, which help in reducing skin irritation, promoting healing, and providing antimicrobial protection. Furthermore, Lidocaine can be included to reduce pain during the tattooing process, making the application experience as comfortable as possible for users.

To enhance user comfort during prolonged operations, the replaceable makeup ink cartridge system incorporates advanced vibration dampening materials within the cartridge. Materials such as silicone or rubber are integrated into the cartridge's structure for their superior vibration-absorbing properties. Additional layers of gel padding around the cartridge housing further mitigate vibrations. A dynamic dampening system, responsive to the intensity of detected vibrations, adjusts accordingly to maintain a steady and comfortable experience. Small isolation mounts or bushings are strategically placed between the cartridge and the pen body to minimize vibration transfer, ensuring smooth operation.

The system may also include an innovative method for ink removal using a saline solution, which operates on the principle of osmosis. This technique involves introducing saline at the site of unwanted tattoo ink to create an osmotic pressure difference that draws out ink particles. Saline cartridges are designed to integrate seamlessly with the existing system, containing solutions that facilitate safe and effective ink removal. Dual-purpose needles capable of handling both ink and saline solutions ensure the system's versatility and functionality. Adjustments to the control system allow precise management of saline application, maintaining high hygiene standards essential for skin procedures. This dual functionality allows the system to not only apply but also selectively remove makeup and tattoos, providing a comprehensive tool for beauty professionals.

In some embodiments of FIGS. 1A-4, a replaceable makeup ink cartridge system is provided which may comprise a distal applicator portion at least partially enclosed by a central gripping portion, wherein: the distal applicator portion comprises a needle cartridge housing, the needle cartridge housing comprises a plurality of apertures disposed therethrough, and the needle cartridge housing encloses a reciprocating slide element comprising a makeup ink cartridge therein; the central gripping portion comprising a tapered outer housing, wherein: the tapered outer housing defines a cavity therein, and the cavity comprises a driving element disposed therein which is in operable communication with the reciprocating slide element; and an applicator actuator in electrical communication with the driving element and electrical or mechanical communication with locking tabs securing the distal applicator portion in place.

In some embodiments of FIGS. 1A-4, the reciprocating slide element is coupled to an applicator needle, the makeup ink cartridge is coupled to a fluid channel, the makeup ink cartridge is fluidically coupled to the applicator needle via the fluid channel, the reciprocating slide element is at least partially disposed through a coiled element, and the reciprocating slide element and the coiled element are surrounded by an encasement medium.

In some embodiments of FIGS. 1A-4, the tapered outer housing comprises an internal aperture, the tapered outer housing comprises an internal peripheral flange defining the internal aperture, and a distal end of the driving element is disposed through the internal aperture.

In some embodiments of FIGS. 1A-4, the tapered outer housing comprises a first housing portion and a second housing portion, the first housing portion is disposed at a proximal end thereof and the second housing portion is disposed at a distal end thereof, the first housing portion comprises a cylindrical shape with no degree of taper, the second housing portion comprises a conical tapered shape, the applicator actuator is disposed through the first housing portion, the locking tabs are disposed into an inner surface of the second housing portion, the first housing portion comprises a battery charging port, the first housing portion comprises a partially removable hatch door, and the partially removable hatch door in an open configuration provides a opening sized to accept the makeup ink cartridge.

In some embodiments of FIGS. 1A-4, a replaceable makeup ink cartridge system is provided which may comprise a distal applicator portion at least partially enclosed by a central gripping portion, wherein: the distal applicator portion comprises a needle cartridge housing, the needle cartridge housing comprises a plurality of apertures disposed therethrough, an applicator needle is disposed through only one of the plurality of apertures, the needle cartridge housing encloses a reciprocating slide element comprising a makeup ink cartridge therein, and the makeup ink cartridge is in fluid communication with the applicator needle via a fluid channel disposed therebetween; the central gripping portion comprising a tapered outer housing, wherein:

the tapered outer housing defines a cavity therein, and the cavity comprises a driving element disposed therein which is in operable communication with the reciprocating slide element; and an applicator actuator in electrical communication with the driving element and electrical or mechanical communication with locking tabs securing the distal applicator portion in place.

In some embodiments of FIGS. 1A-4, a replaceable makeup ink cartridge system is provided which may comprise a distal applicator portion at least partially enclosed by a central gripping portion, wherein: the distal applicator portion comprises a needle cartridge housing, the needle cartridge housing comprises a plurality of apertures disposed therethrough, and the needle cartridge housing encloses a reciprocating slide element comprising a makeup ink cartridge therein; the central gripping portion comprising a tapered outer housing, wherein: the tapered outer housing defines a cavity therein, and the cavity comprises a driving element disposed therein which is in operable communication with the reciprocating slide element; and an applicator actuator in electrical communication with the driving element and electrical or mechanical communication with locking tabs securing the distal applicator portion in place, wherein: the applicator actuator comprises a slide handle, the slide handle translates iteratively along a translation track between a plurality of serial positions, and when the slide handle is placed in a release position, the slide handle is disposed adjacent a buffer spring, and when the buffer spring is displaced by the slide handle, the locking tabs are released from a locked state and placed into an unlocked state.

The specification and drawings are to be regarded in an illustrative rather than a restrictive sense. However, it will be evident that various modifications and changes may be made thereunto without departing from the broader spirit and scope of the invention as set forth in the claims. Other variations are within the spirit of the present disclosure. Thus, while the disclosed techniques are susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

All features disclosed in the specification, claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise.

Throughout this disclosure, the phrase ‘modularly coupled’ and similar terms and phrases are intended to convey that any element of a given class of elements may be coupled to another given element and vice versa with equal effect. For example, any extension cord of a plurality of extension cords may be modularly coupled to another extension cord and vice versa with equal effect. Further, throughout this disclosure, the phrase ‘removably coupled’ and similar terms and phrases are intended to convey that a given element may be iteratively coupled to and removed from another given element as desired. For example, a male plug of a first extension cord may be removably coupled to a female plug of a second extension cord as desired.

The use of the terms “a,” “an,” “the,” and similar referents in the context of describing the disclosed embodiments (especially in the context of the following claims) are to be construed to cover both the singular and the plural, unless otherwise indicated or clearly contradicted by context. The terms “comprising,” “having,” “including,” and “containing” are to be construed as open-ended terms (i.e., meaning “including, but not limited to,”) unless otherwise noted. The term “coupled” or “connected,” where unmodified and referring to physical connections, is to be construed as partly or wholly contained within, attached to, or joined together, even if there is something intervening. Recitation of ranges of values are merely intended to serve as a shorthand method of referring individually to each separate value falling within the range, unless otherwise indicated and each separate value is incorporated into the specification as if it were individually recited. The use of the term “set” (e.g., “a set of items”) or “subset” unless otherwise noted or contradicted by context, is to be construed as a nonempty collection comprising one or more members. Further, unless otherwise noted or contradicted by context, the term “subset” of a corresponding set does not necessarily denote a proper subset of the corresponding set, but the subset and the corresponding set may be equal.

Conjunctive language, such as phrases of the form “at least one of A, B, and C,” or “at least one of A, B and C,” is understood with the context as used in general to present that an item, term, etc., may be either A or B or C, or any nonempty subset of the set of A and B and C, unless specifically stated otherwise or otherwise clearly contradicted by context. For instance, in the illustrative example of a set having three members, the conjunctive phrases “at least one of A, B, and C” and “at least one of A, B and C” refer to any of the following sets: {A}, {B}, {C}, {A, B}, {A, C}, {B, C}, {A, B, C}. Thus, such conjunctive language is not generally intended to imply that certain embodiments require at least one of A, at least one of B and at least one of C each to be present. In addition, unless otherwise noted or contradicted by context, the term “plurality” indicates a state of being plural (e.g., “a plurality of items” indicates multiple items). The number of items in a plurality is at least two, but can be more when so indicated either explicitly or by context.

The use of any examples, or exemplary language (e.g., “such as”) provided, is intended merely to better illuminate embodiments of the invention and does not pose a limitation on the scope of the invention unless otherwise claimed. No language in the specification should be construed as indicating any non-claimed element as essential to the practice of the invention.

Embodiments of this disclosure are described, including the best mode known to the inventors for carrying out the invention. Variations of those embodiments may become apparent to those of ordinary skill in the art upon reading the foregoing description. The inventors expect skilled artisans to employ such variations as appropriate and the inventors intend for embodiments of the present disclosure to be practiced otherwise than as specifically described. Accordingly, the scope of the present disclosure includes all modifications and equivalents of the subject matter recited in the claims appended hereto as permitted by applicable law. Moreover, although above-described elements may be described in the context of certain embodiments of the specification, unless stated otherwise or otherwise clear from context, these elements are not mutually exclusive to only those embodiments in which they are described; any combination of the above-described elements in all possible variations thereof is encompassed by the scope of the present disclosure unless otherwise indicated or otherwise clearly contradicted by context.

All references, including publications, patent applications, and patents, cited are hereby incorporated by reference to the same extent as if each reference were individually and specifically indicated to be incorporated by reference and were set forth in its entirety.

1. A replaceable makeup ink cartridge system, comprising:
a distal applicator portion at least partially enclosed by a central gripping portion, wherein:
the distal applicator portion comprises a needle cartridge housing,
the needle cartridge housing comprises a plurality of apertures disposed therethrough, and
the needle cartridge housing encloses a reciprocating slide element comprising a makeup ink cartridge therein;
the central gripping portion comprising a tapered outer housing, wherein:
the tapered outer housing defines a cavity therein, and
the cavity comprises a driving element disposed therein which is in operable communication with the reciprocating slide element; and
an applicator actuator in electrical communication with the driving element and electrical or mechanical communication with locking tabs securing the distal applicator portion in place.

2. The replaceable makeup ink cartridge system of claim 1, wherein the reciprocating slide element is coupled to an applicator needle.

3. The replaceable makeup ink cartridge system of claim 2, wherein the makeup ink cartridge is coupled to a fluid channel.

4. The replaceable makeup ink cartridge system of claim 3, wherein the makeup ink cartridge is fluidically coupled to the applicator needle via the fluid channel.

5. The replaceable makeup ink cartridge system of claim 4, wherein the reciprocating slide element is at least partially disposed through a coiled element.

6. The replaceable makeup ink cartridge system of claim 5, wherein the reciprocating slide element and the coiled element are surrounded by an encasement medium.

7. The replaceable makeup ink cartridge system of claim 1, wherein the tapered outer housing comprises an internal aperture.

8. The replaceable makeup ink cartridge system of claim 7, wherein the tapered outer housing comprises an internal peripheral flange defining the internal aperture.

9. The replaceable makeup ink cartridge system of claim 8, wherein a distal end of the driving element is disposed through the internal aperture.

10. The replaceable makeup ink cartridge system of claim 1, wherein the tapered outer housing comprises a first housing portion and a second housing portion.

11. The replaceable makeup ink cartridge system of claim 10, wherein the first housing portion is disposed at a proximal end thereof and the second housing portion is disposed at a distal end thereof.

12. The replaceable makeup ink cartridge system of claim 11, wherein the first housing portion comprises a cylindrical shape with no degree of taper.

13. The replaceable makeup ink cartridge system of claim 12, wherein the second housing portion comprises a conical tapered shape.

14. The replaceable makeup ink cartridge system of claim 13, wherein the applicator actuator is disposed through the first housing portion.

15. The replaceable makeup ink cartridge system of claim 13, wherein the locking tabs are disposed into an inner surface of the second housing portion.

16. The replaceable makeup ink cartridge system of claim 13, wherein the first housing portion comprises a battery charging port.

17. The replaceable makeup ink cartridge system of claim 13, wherein the first housing portion comprises a partially removable hatch door.

18. The replaceable makeup ink cartridge system of claim 17, wherein the partially removable hatch door in an open configuration provides a opening sized to accept the makeup ink cartridge.

19. A replaceable makeup ink cartridge system, comprising:
a distal applicator portion at least partially enclosed by a central gripping portion, wherein:
the distal applicator portion comprises a needle cartridge housing,
the needle cartridge housing comprises a plurality of apertures disposed therethrough,
an applicator needle is disposed through only one of the plurality of apertures,
the needle cartridge housing encloses a reciprocating slide element comprising a makeup ink cartridge therein, and
the makeup ink cartridge is in fluid communication with the applicator needle via a fluid channel disposed therebetween;
the central gripping portion comprising a tapered outer housing, wherein:
the tapered outer housing defines a cavity therein, and
the cavity comprises a driving element disposed therein which is in operable communication with the reciprocating slide element; and
an applicator actuator in electrical communication with the driving element and electrical or mechanical communication with locking tabs securing the distal applicator portion in place.

20. A replaceable makeup ink cartridge system, comprising:
a distal applicator portion at least partially enclosed by a central gripping portion, wherein:
the distal applicator portion comprises a needle cartridge housing,
the needle cartridge housing comprises a plurality of apertures disposed therethrough, and
the needle cartridge housing encloses a reciprocating slide element comprising a makeup ink cartridge therein;
the central gripping portion comprising a tapered outer housing, wherein:
the tapered outer housing defines a cavity therein, and
the cavity comprises a driving element disposed therein which is in operable communication with the reciprocating slide element; and
an applicator actuator in electrical communication with the driving element and electrical or mechanical communication with locking tabs securing the distal applicator portion in place, wherein:
the applicator actuator comprises a slide handle,
the slide handle translates iteratively along a translation track between a plurality of serial positions, and
when the slide handle is placed in a release position, the slide handle is disposed adjacent a buffer spring, and when the buffer spring is displaced by the slide handle, the locking tabs are released from a locked state and placed into an unlocked state.
